(19) United States
(12) Patent Application Publication
(10) Pub. No.: US 2013/0210139 A1
(43) Pub. Date: Aug. 15, 2013

(54) PROTEOLIPIDBEADS AND METHOD OF USE THEREOF

(71) Applicant: Research Foundation of the City University of New York, (US)

(72) Inventors: M. Lane Gilchrist, New York, NY (US); Bin He, New York, NY (US)

(73) Assignee: Research Foundation of the City University of New York, New York, NY (US)

(21) Appl. No.: 13/769,145

(22) Filed: Feb. 15, 2013

Related U.S. Application Data
(60) Provisional application No. 61/599,199, filed on Feb. 15, 2012.

(51) Int. Cl. C12N 5/071

(52) U.S. Cl. CPC C12N 5/0662
USPC 435/366; 435/325

(57) ABSTRACT

The subject matter disclosed in this specification pertains to the transfer of compounds of interest into a target biological cell. Specifically, discrete particles that are surrounded in three dimensions with phospholipid bilayers are provided wherein the compound of interest (e.g.) a protein are free to laterally move within the bilayer. The particles may be embedded within a hydrogel matrix. In some embodiments, stem cells are co-cultured in the hydrogel matrix to facilitate the absorption of the compound of interest.

### CROSS-REFERENCE TO RELATED APPLICATIONS

This application claims priority to and the benefit of U.S. provisional patent application Ser. No. 61/599,199 (filed Feb. 15, 2012) which application is incorporated herein by reference in its entirety.

### STATEMENT REGARDING FEDERALLY FUNDED RESEARCH OR DEVELOPMENT

This invention was made with government support under contract no. 41341-01-30 awarded by the National Institute of Health (NIH). The government has certain rights in the invention.”

### FIELD OF THE INVENTION

This invention relates, in one embodiment, to the transfer of compounds of interest into a target biological cell. Specifically, discrete particles that are surrounded in three dimensions with phospholipid bilayers are provided wherein the compound of interest (e.g.) a protein are free to laterally move within the bilayer.

### BACKGROUND

Cell culture provides a vital tool for studying cellular behavior. However, traditional cell culture methods generally establish a very artificial environment for cells, wherein normal cell behavior is disrupted. It would be advantageous to develop new cell culture methods that more accurately mimic physiological conditions. In the body, cells reside in tissue-specific niches, physically constricted in an extracellular matrix where they are in close contact with neighboring cells as well a soluble mix of chemokines, cytokines, growth factors, and other species. It would be advantageous to develop a culturing system that mimics the cell niche and which would allow researchers to recreate physiologically realistic conditions so that the spatial and temporal signaling that directs cell physiology in vivo can be studied. Such a culturing system could also serve as a platform for cell engineering and propagation.

Membrane proteins play a vital role in cellular function. Membrane proteins are involved in all aspects of cell physiology, including cell-to-cell communication and adhesion, transport of materials in and out of the cell, and cellular sensing of the external environment. Accordingly, there has been an intense research effort to develop artificial systems that mimic the cell membrane. The goal of such efforts is typically to create a system where membrane constituents can move laterally in the phospholipid bilayer, recapitulating the in vivo environment. Supported membranes are phospholipid bilayers attached or adsorbed onto solid substrates which can be used to study various aspects of membrane biology. Many types of supported membrane systems have been developed, including bilayers attached to planar surfaces, sometimes patterned into arrays of discreet areas.

Numerous variations of the supported membrane concept have been developed. Membranes can be adsorbed directly onto substrates, however this limits the desired lateral mobility of membrane proteins. Membranes can also be tethered to “polymer cushions,” which separate the membrane from the substrate and allow for some mobility of the membrane proteins.

Membranes can also be tethered to moieties that act to secure the membrane to the substrate and, at the same time, act as spacers which create a layer of solute between the membrane and the substrate of sufficient thickness that the substrate will not interfere with movement and function of proteins in the membrane.

Stem cells hold great promise as a source of therapies for the regeneration of injured, aged, or diseased tissues. A massive research effort is underway to better understand the factors that influence stem cell differentiation pathways and maintenance of pluripotency. It would be advantageous to develop systems which interact with stem cells in physiologically realistic conditions, in order to better understand the signaling mechanism which control stem cell fate. Such a system could also be utilized to manipulate stem cell fate in order to propagate stem cells or direct their differentiation.

In order to study protein function and cell physiology, it is often advantageous to introduce new proteins into cells, including cell membrane proteins. This can be accomplished using genetic means, with exogenous genetic material coding for a protein of interest being either transiently expressed or integrated into the genome of the cell. However, using these genetic tools brings many complications, including disruptions associated with the introduction of genetic material, ensuring proper regulation of gene expression, ensuring proper protein folding, etc. Alternative non-genetic techniques, such as microinjection, can be used to introduce proteins into cells, but these techniques damage cells and create artifacts. It would advantageous to develop a system of introducing membrane proteins and other proteins to cells in such a way that does not overly disrupt the cell's normal function.

### SUMMARY OF THE INVENTION

The subject matter disclosed in this specification pertains to the transfer of compounds of interest into a target biological cell. Specifically, discrete particles that are surrounded in three dimensions with phospholipid bilayers are provided wherein the compound of interest (e.g.) a protein are free to laterally move within the bilayer. The particles may be embedded within a hydrogel matrix. In some embodiments, stem cells are co-cultured in the hydrogel matrix to facilitate the absorption of the compound of interest.

### BRIEF DESCRIPTION OF THE DRAWINGS

The present invention is disclosed with reference to the accompanying drawings, wherein:

FIG. 1 is a schematic diagram of a method of construction an exemplary proteolipobead hydrogel system;

FIG. 2 is a graph depicting cellular adhesion to an exemplary proteolipobead;

FIG. 3 is a graph depicting cell viability;

FIG. 4 is a graph of proteolipobead contact area;

FIG. 5 depicts proteolipobead contacts; and

FIGS. 6A, 6B, 6C and 6D depict images of stem cells and beads.

Corresponding reference characters indicate corresponding parts throughout the several views. The examples set out herein illustrate several embodiments of the invention but should not be construed as limiting the scope of the invention in any manner.

### DETAILED DESCRIPTION

The invention described herein uses supported membranes on substrates, specifically, the use of phospholipid bilayers on discrete particles to provide proteolipobeads. The invention encompasses novel proteolipobead compositions and methods of using proteolipobeads.

The term proteolipobead (PLB), as used herein, refers to a composition of matter that comprises a discreet particle which supports a lipid bilayer. The supported lipid bilayers of the invention can be of any construction which allows for free lateral movement of a protein embedded the bilayer. Typically, this lateral mobility is preserved by separating the bilayer from the discreet particle by one of two means. One means of separation is the use of a “polymer cushion” between the discreet particle and the bilayer. In one embodiment, the means of separation is a hydrated hydrogel material. A second means of separation is the use of a tether between the bilayer and the discreet particle of such length to create adequate separation between the discreet particle and the bilayer for mobility of proteins embedded in the bilayer.

### The Discrete Particle

The function of the discrete particle is to provide a stable support for the membrane bilayer, which creates a body having discreet size and shape. Accordingly, any material compatible with polymer cushion coating or tethering technologies can be used as the discrete particle. Compatible materials will typically consist of a solid material which is functionalized for conjugation chemistry. Examples of types currently in use in these studies are 3 micron (Dynal magnetic/Invitrogen), 5 and 20 micron silica (Bangs Laboratories; Kisker Biotech), and 30 micron glass (Polysciences, Inc). For example, silica, glass, organic molecules, metallic molecules, and hybrids thereof can be functionalized with any number of moieties (e.g. carboxylic acid, amines) which allow for the facile conjugation attachment of polymer cushions or tethering molecules.

The discrete particle size can range from 100 nanometers to 100 microns. If it is desired that the bead interact with a specific cell type, the size of the particle can be tailored to match the desired interaction. For example, if it is desired that the target cell incorporate the bead within itself, the size of the discrete particle should be smaller than the cell, for example in the range of 1-5 microns. If it is intended that the bead act as a cellular mimic, it may be roughly the size of the cell being mimicked. In one embodiment, the discrete particle has a diameter between about 10 micrometers and about 100 micrometers.

The discrete particle can also serve secondary functions, such as facilitating sorting, purification, or measurement of electronic properties. Accordingly, materials such as fluorescently or magnetically coded particles, magnetic and paramagnetic particles, and semiconductors can be utilized for the discrete particle in order to facilitate processes such as flow cytometry, magnetic separation, and detection of changing electric fields.

### The Lipid Bilayer

The lipids utilized in the bilayer can be any phospholipid capable of forming bilayer membrane structures, including phosphatidic acid, phosphatidylethanolamine, phosphatidylcholine, phosphatidylserine, phosphatidylinositol, phosphatidylinositol phosphate, phosphatidylinositol bisphosphate, phosphatidylinositol triphosphate, a diglyceride phospholipid and mixtures thereof. Cell membranes from living cells can be utilized as well.

The lipid bilayer may further comprise a non-polar organic molecule that alters the structure of the lipid bilayer. For example, cholesterol may be used.

### The Compound

Proteins, peptides, and other compounds of interest (e.g. a nucleic acid) can be included in the bead. Full-sequence proteins isolated from living cell membranes or expressed recombinantly can be used. Alternatively or additionally, the extracellular domains of many membrane proteins have been identified, and these can be created through standard recombinant techniques. Many such extracellular domains, for example, those of the Jagged and Notch proteins, are available commercially. These extracellular domains can be anchored to the lipid bilayer by a number of means. For example, peptides can be anchored to glycolipid anchors such as those described in U.S. Publication No. 2009/0226960, which describes an anchoring system based on the glycolipid glycosylphosphatidylinositol. Methods of lipidating proteins so that they may be anchored to membranes are available. Anchoring system based on cationic polymers displaying secondary amines are also available. Peptides constituting extracellular domains can also be attached to membrane lipids by functionalizing the peptide and lipids with complementary binding agents such as avidin-biotin or nickel-nitrilotriacetic acid/HIS tags.

In one embodiment of the invention, the beads are functionalized with at least one cell adhesion molecule. Cell adhesion molecules include any peptide or other composition which will bind to moieties present in or on the membrane of a living cell. A bead functionalized with a cell adhesion molecule means the cell adhesion molecule is anchored to or embedded within the supported membrane on the bead in such a way that it is presented to cells coming in contact with the bead. Exemplary cell adhesion molecules include integrins, selectins, and cadherins. Cell adhesion molecules further include antibodies or antibody fragments which will bind to epitopes on cell membranes. The quantity of ligands displayed on the surface of an individual beads can be quantified with flow cytometric methods. Furthermore, fluorescence activated cell sorting can be used to separate out the beads that have the desired quantity of ligands displayed.

### Lateral Mobility

The lipid bilayers are associated with the discreet particles such that the lateral mobility is preserved. One means of separation is the use of a “polymer cushion” between the discreet particle and the bilayer. In one embodiment, the means of separation is a hydrated hydrogel material. A second means of separation is the use of a tether between the bilayer and the discreet particle of such length to create adequate separation between the discreet particle and the bilayer for mobility of proteins embedded in the bilayer.

Various methods of supporting lipid bilayers on polymer cushions are known in the art, utilizing a wide variety of materials as the cushion, including dextran, cellulose and polyelectrolytes. In these examples, the discreet particle is coated with the polymer materials. In other embodiments, the discreet particle itself acts as the cushion, for example one may use cross-linked organic polymers as described in U.S. Pat. No. 7,883,648.

Tethering moieties such as thiolipids, polyethylene glycol-lipids, oligopeptides and bacteriorhodopsin conjugates may be used. The surfaces are functionalized with aminosilanes when tethering or other surface manipulation is desired. For added stability tethering to the biomembrane may be accomplished using two methods: 1) bridging from surface amines to amino-lipid head groups using an amine-reactive homobifunctional crosslinker (NHS-PEG-NHS) or 2) bridging from biotin linked to surface amines to biotin-labeled lipids using streptavidin. The encoding of the assemblies is realized by either size encoding, by spectrally encoding the surface using labeled streptavidin or spectrally encoding the lipid bilayer with fluorescent tracer dyes.

### Cell Culture

The invention further encompasses the use of the beads described herein in cell culture systems. The beads can be utilized in standard two-dimensional cell cultures, solution cultures, or three-dimensional culturing systems. Three-dimensional culturing systems include crosslinked hydrogel systems. Exemplary hydrogels are those composed of polyethylene glycol, carboxymethlycelllose, self-assembling peptide matrices or natural polymers such as collagen-I.

The cell culturing systems described above can be seeded with any number of cell types, including stem cells, blood cells, tissue cells, or cancer cells. Cells from any species can be utilized, including humans, animals, plants, and single-celled organisms such as bacteria or yeast. Stem cells may be embryonic stem cells or adult stem cells, including hematopoietic, mesenchymal, endothelial, and neural stem cells. Cells and beads can be seeded in specific proportions to facilitate the desired number of cell-bead contacts.

It has been discovered, unexpectedly, that beads co-cultured with cells can fuse with the cells. Without being limited by any one theory of operation, it is hypothesized that the inclusion of cell adhesion molecules on the surface of the beads facilitates the fusion of the cell membrane with the supported membrane on the beads. Based upon this theory of operation, the invention encompasses the use of cell adhesion molecules in systems other than beads, including liposomes, vesicles, and membranes supported on planar surfaces.

The fusion of beads and living cells creates an opportunity to deliver materials to living cells in a non-genetic manner that avoids the cell damage and artifacts created by techniques such as microinjection. Beads can be loaded with proteins (both integral membrane proteins and membrane-anchored proteins), lipid-soluble materials such as dyes. The internal space between the supported membrane and the bead substrate can also be loaded with water soluble compounds by including these compounds in the solution at the time the membrane is formed. Dyes, fluorescent markers, drugs, nucleic acid constructs and other water-soluble compounds can be included. These beads can then be contacted with living cells and when fusion occurs, the materials incorporated in the bead will be delivered to the living cell.

Delivery rates can be enhanced by utilizing beads as intermediates between a source of compounds and the cell. For example, beads can be immobilized on micropipette tips, nanoporous filters such as anodized aluminum oxide or immobilized in contact with micropipette tips. When cells fuse with the beads, a positive flow can be effected in the micropipette or filter to deliver solutions to the living cell via the immobilized beads through the biomembrane or the underlying solution trapped beneath the biomembrane surface.

If the bead is smaller than the cell, cell fusion with the bead will result in the bead being engulfed and essentially incorporated into the cell. In such event, the bead substrate can be used to impart new properties to the cell. For example, labeled substrates will allow imaging, tracking, and sorting of cells. Magnetic and paramagnetic substrates can facilitate purification of cells from culture materials.

### Examples

The core elements for ligand display are proteolipobeads fashioned from commercially available microspheres of various sizes and materials. The types currently used in these studies are 20-50 micron and 30 micron glass size standards (Duke Scientific, Inc).

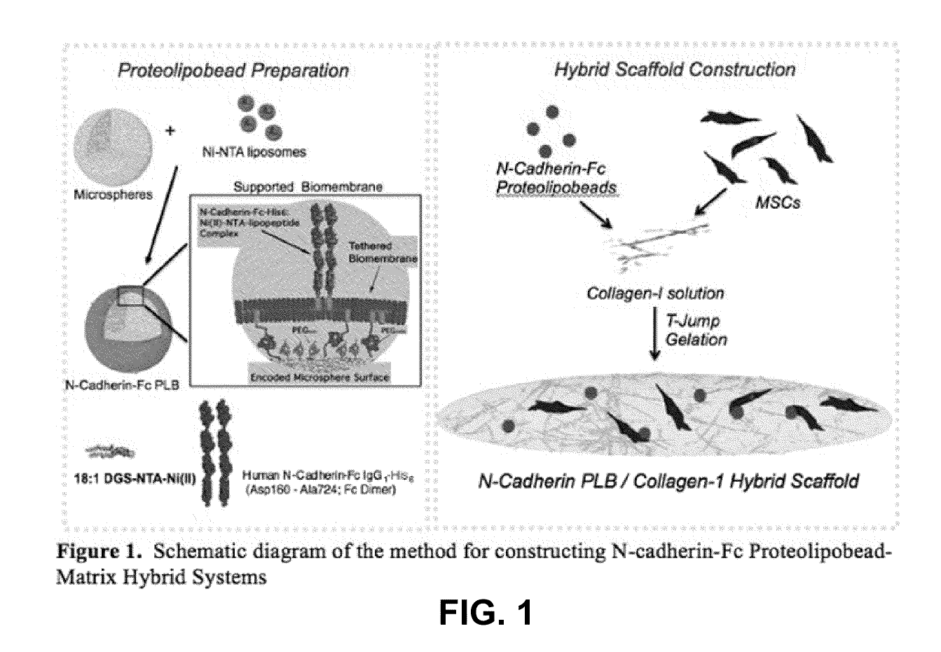

FIG. 1 outlines one method for PLB formation. Ni²⁺-NTA-PE containing lipid formulations were used with DiD added as a lipid tracer and liposomes formed by sonication were fused with the microspheres at liposome to microsphere surface area in greater than five-fold excess. The coverage was examined with confocal microscopy. To quantify the surface display we have employed flow cytometry in concert with FITC calibration beads and anti-N-Cadherin-FITC conjugates to examine the N-cadherin densities on the proteolipobead surfaces.

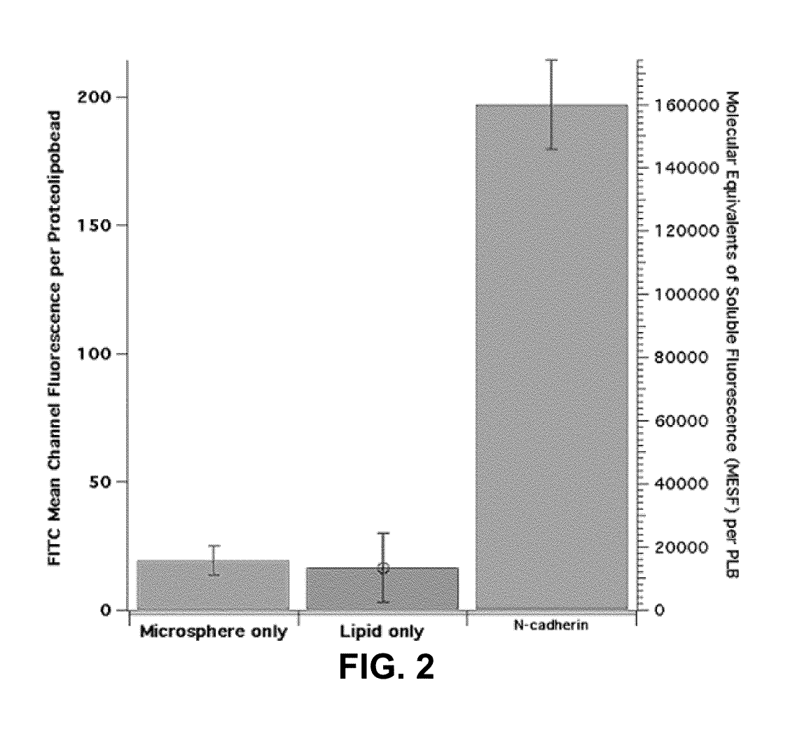

FIG. 2 displays a bar chart of the characterization of ligand display monitored by binding anti-N-cadherin-FITC to 30 micron N-cad-Fc-His₆ PLBs and controls. We choose microspheres without surface modification as negative control, as the left bar, Ni²⁺NTA-DGS PLBs not complexed with N-Cadherin-Fc-His₆ were displayed as positive control in the middle bar. The right bar is from N-Cadherin-Fc-His₆-Ni²⁺NTA-DGS PLBs. All were incubated first in 0.1% BSA blocking and then at >2-fold excess Anti-N-Cadherin-FITC concentration, incubating at 4° C. overnight. To analyze N-Cadherin coverage on PLBs, we applied anti-human N-Cadherin fluorecein to the bare beads (left column), the lipid bilayer fused beads (middle column) and N-Cadherin PLBs (right column) in this chart. FITC MESF kit was addressed to quantify N-Cadherin coverage on microspheres. The left axis is given in Molecular Equivalents of Soluble Fluorescence per PLB, obtained from fluorescein calibration beads from Bangs Laboratories run at the same settings. The detected N-cadherin displayed per PLB is 10 fold greater in the N-Cadherin PLBs (right bar) than in the case of the negative controls (left and middle bars). Under these conditions, based on the anti-N-cadherin-FITC F/P ratio the N-Cadherin-Fc-His₆ dimer surface concentration was estimated to be approximately 28 ligands per square micron.

To analyze ligand display within the scaffold we constructed the microsphere/collagen-I matrix without interference of stem cells to analyze the N-Cadherin ligand distributions on the proteolipobeads. Analysis of the lipid bilayer coverage of a random sampling of the equatorial Z sections of n=10 N-cad-Fc-PLBs gave lipid coverage percentages of 84±0.02% and the anti-N-Cad-FITC coverage percentage was 83±0.03%, indicating that the PLBs are largely intact after collagen-I gelation. The colocalization of lipid and the N-cadherin-Fc was high, with a Mander's overlap colocalization coefficient of 0.82±0.02, obtained from a random sampling of n=10 N-Cadherin PLBs within the collagen-I gel. The negative control sample with no added N-Cadherin-Fc-His₆ showed negligible Anti-N-Cad-FITC signal levels at the same detector settings (data not shown).

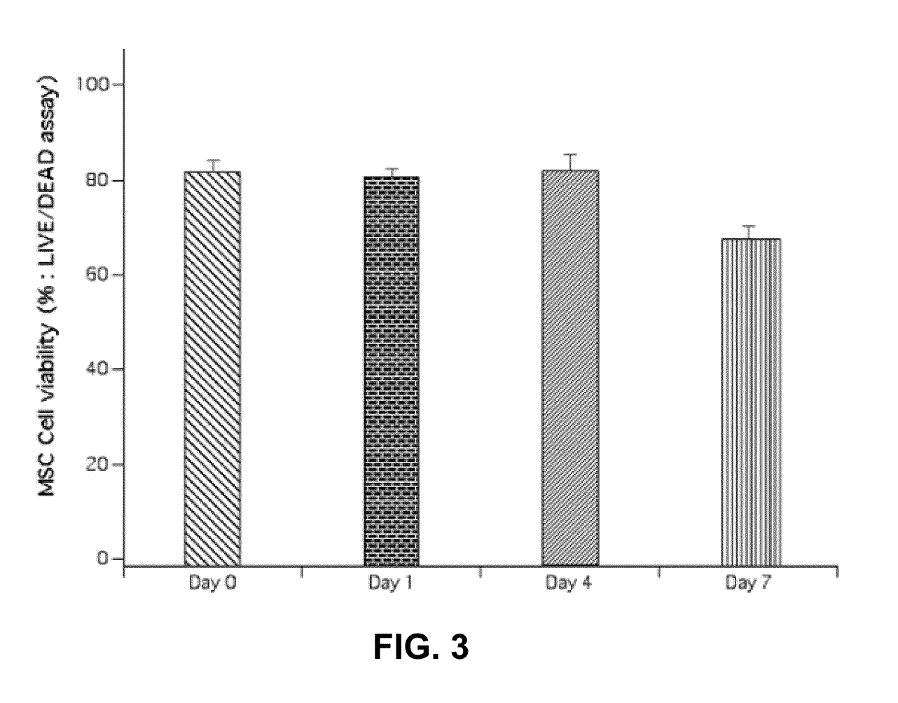

Additional studies were concerned with investigating the N-cadherin proteolipobead properties after collagen gelation, and probing the interaction between N-cadherin proteolipobeads and human mesenchymal stem cells (hMSCs) by monitoring MSC viability. At day 1 the hMSC cell viability was over 80% and decreased to 67.5% by Day 7 as obtained from the Calcein-AM/Ethidium homodimer LIVE/DEAD assay, thus high cell viability was evidenced (FIG. 3). Numerous cell-to-PLB interactions were found in the collagen-cell-PLB hybrid system at day 1, we describe these interactions further below.

### Cadherin Proteolipobead/Collagen-I/hMSC 3D Constructs

At the onset of the experiment a subset of the constructs in culture were examined with CLSM under live-cell conditions. The three dimensional constructions of representative hMSCs in microsphere-collagen-I hybrid scaffolds were obtained. The stem cells were growing in the collagen matrix and formed the characteristic “spindle” shape of hMSCs. The assemblies were loaded with N-Cadherin-Fc-His₆, in complexes with the His₆ binding “receptor” ligand Ni-NTA-PE.

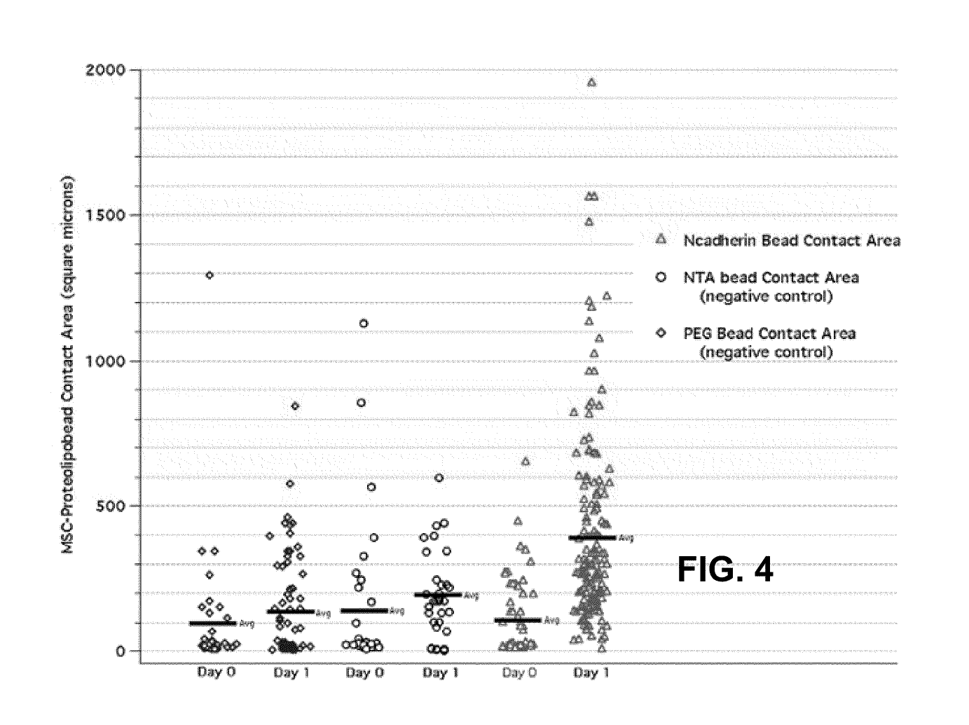

In further studies, 20-micron silica N-cadherin-proteolipobeads were combined in a collagen-I scaffold and seeded with hMSCs. FIG. 4 scatter chart shows the interaction area between hMSCs and microsphere assemblies within the 3D matrix. The hMSC-microsphere interaction area goes up >3.8-fold from day 0 to day 1 for N-cadherin proteolipobeads when the cell processes form; (negative) control microspheres included in the same 3D construct that were passivated with PEGylated surfaces (no lipid, no N-cadherin) gave 2-fold less interaction area in Day 1. In this case, the increase of cell to PLB interaction area is consistent with the effect of the adhesion protein N-Cadherin displayed on the PLB surface.

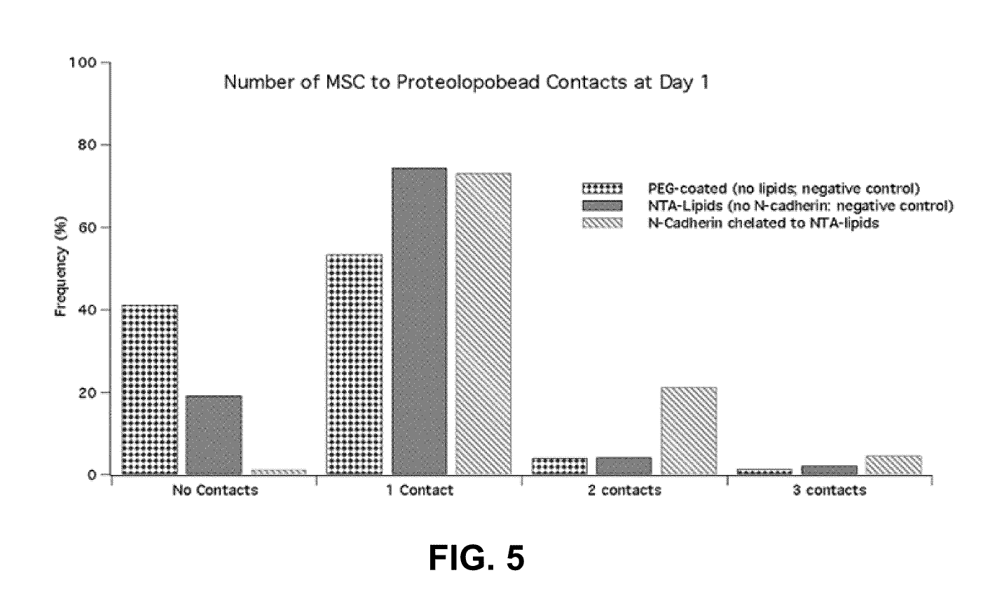

FIG. 5 shows a plot of the number of contacts per bead for Day 0 versus Day 1 with negative controls. Our finding is that there are substantially more contacts per N-cadherin proteolipobead than in the negative controls. Furthermore, few of the N-cadherin PLBs have no contacts, unlike in the two negative control cases. The formation of the MSC-Collagen-1 3D matrix around the proteolipobead assemblies did not appreciably disturb the lipid bilayers, as the lipid tracer fluorescence coverage did not change in statistically relevant amounts before and after gelation.

Further studies were conducted to visualize the proteolipobead-displayed N-cadherin engaged in hMSC interactions in situ. We studied 3D CLSM reconstructions of hMSCs in N-cadherin proteolipobead/Collagen-I 3D constructs at 63× magnification. The assemblies with proteolipobead-displayed N-Cadherin were localized with Anti-N-Cadherin-phycoerytherin and the hMSCs stained with Calcein-AM. Analysis at 63× magnification of 17 randomly selected N-Cad-PLBs (and n=43 interacting MSCs) at day 1 gave an average area per contact value of 369.8±31 micron/cell contact. The N-cadherin (anti-N-cadherin-PE detected) average percent coverage from to be 80.6±10.8%, slightly less than the percent microsphere lipid coverage. The PLBs were largely intact, although some evidence for lipid bilayer loss or damage was evidenced.

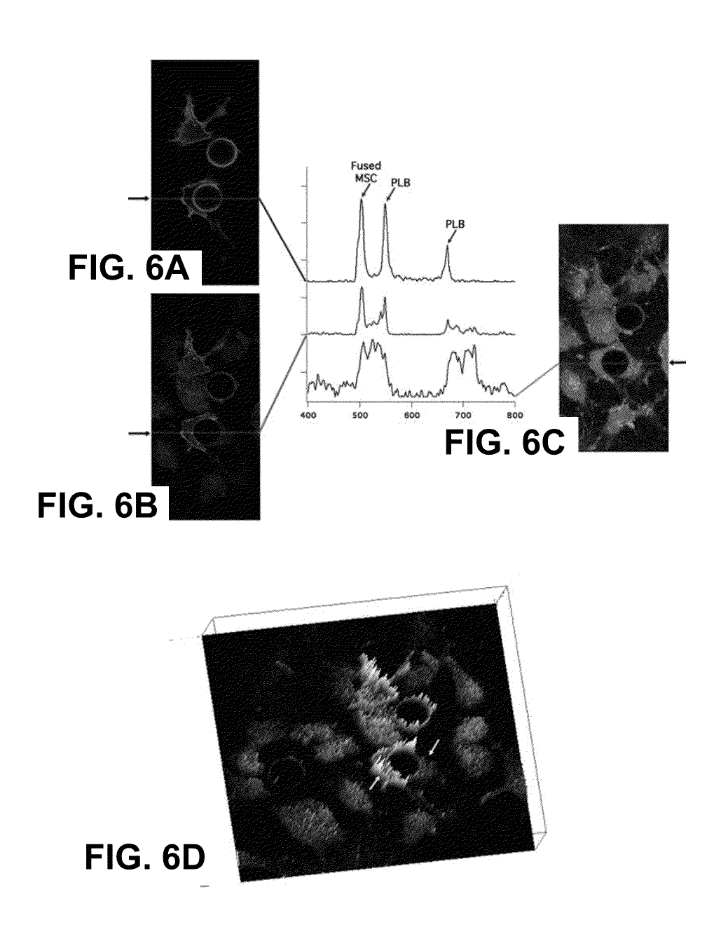

In a fraction of the MSCs interacting with the PLBs (4 out of 43 MSCs), images consistent with PLB-to-MSC biomembrane fusion were evidenced. Analysis of a representative apparent PLB-MSC fusion highlighting delivery of N-Cadherin to the plasma membrane of live MSCs is shown in FIGS. 6A to 6D. FIGS. 6A, 6B and 6C are individual Z-sections in the same XY plane (DiD lipid tracer (6A); anti-Ncadherin-PE; (6B) and Calcein-AM: 6C). Image line profiles are extracted as indicated by the horizontal arrows and displayed as traces in the central inset (DiD: top; Anti-Ncadherin-PE: middle; Calcien-AM: bottom), the X axis indicates voxel number. FIG. 6D displays the same Z-section in 3D with the intensity axis indicating where high levels N-cadherin is stained with anti-Ncadherin-PE. The left arrow points at the fused MSC and the right side arrow points at a MSC interacting with the PLB that did not fuse. It is clear from FIGS. 6A to 6D that MSCs interacting strongly with the same PLB assembly do not show similar loading of DiD tracer and N-Cadherin-Fc-His6 display at their cell periphery, essentially serving as negative controls to fusion and N-cadherin protein transfer. In multiple hybrid constructs, containing hundreds of cells, we did not find any MSCs containing DiD that were not directly in contact with N-Cadherin PLBs. This apparent fusion process and transfer of DiD probe and N-Cadherin is consistent with lateral lipid mobility in the PLB structures.

### Materials and Methods

### Materials

1-palmitoyl-2-oleoyl-sn-glycero-3-phosphatidylcholine (POPC), (Egg, Chicken) 99%, 1,2-di-(9Z-octadecenoyl)-sn-glycero-3-RN-(5-amino-1-carboxypentyl)iminodiacetic acid)succinyl] (nickel salt) (Ni-NTA-PE) were purchased from Avanti Polar Lipids (Alabaster, Ala.). 1,1′-dioctadecyl-3,3,3′,3′-tetramethylindodicarbocyanine,4-chlorobenzenesulfonate Salt (‘DiD’; DiIC₁₈(5)) was purchased from Invitrogen. The hMSC was a gift from Dr. Sihong Wang's lab (Biomedical Engineering Department, CCNY). The hMSC culture medium was composed from MSCBM basal medium 440 ml (stored at 4° C.), SingleQuots Cryovials 1 ml, MSGS 60 ml, L-Glutamine and GA-1000 (stored at −20° C.). Recombinant Human N-Cadherin Fc Chimera ((N-terminus)N-Cadherin:Asp160-Ala724)-IEGRMD-Human IgG1:(Pro100-Lys330)-His6(C-terminus)) monoclonal anti-human N-Cadherin Propeptide-Phycoerythrin (PE-conjugated antibody) and monoclonal Anti-human N-Cadherin Propeptide-Fluorescein (FITC-conjugated antibody) were obtained from R&D Systems. Rat-tail collagen type I was purchased from Becton Dickenson Laboratories.

### Proteolipobead Synthesis

The lipid mixture was made of POPC, cholesterol and NiNTA-DSPE in different molar proportions. POPC/Cholesterol/DiD in 0.8:0.198:0.002 in molar proportions, POPC/Cholesterol/NiNTA-DSPE/DiD in 0.7:0.198:0.1:0.002 in molar proportions and POPC/Cholesterol/NiNTA-DSPE/DiD in 0.75:0.198:0.05:0.002. These formulations were mixed using chloroform and dried overnight under vacuum, forming a thin film in approximately 2 mg per vial. 2 ml PBS buffer was then introduced to make the lipids concentration 1 mg/ml and the vial vortexed for 1 min. the lipids were refrozen at −20° C. solid and thaw in 4° C. water before an intense 15 min probe sonication was conducted in icy water. 30 or 30-50 micron glass beads were incubated by Ni-NTA-PE lipid for 30 minutes with occasionally stir. A lipid bilayer was formed as a thin membrane in fluidic form where the individual lipid molecules are constantly in motion around the 30 micron silica beads. This step was followed by three time rinses with PBA buffer to get rid of access lipid as background signals. Human N-Cadherin-Fc-His₆ was then introduced onto the surface of the Ni²⁺-NTA-DSPE lipobead system in two-fold excess in Ca²⁺-free PBS buffer. The transmembrane domain of N-Cadherin Fc His₆ was chelated to the lipid bilayer of the LBs after one hour incubation at 4° C., giving N-Cadherin proteolipobeads. The extracellular domain of N-Cadherin used contain the calcium-binding domains, and have shown functional homotypic binding in previous studies.

### Proteolipobead Characterization via Flow Cytometry and Confocal Microscopy

To test the N-Cadherin coverage of the proteolipobeads, we applied monoclonal anti-human N-Cadherin-phycoerythrin and -fluorecein separately. Anti-human N-Cadherin phycoerythrin gives red color in confocal sequential 3D scanning, which helped us distinguish the live stem cell stain (Calcien-AM, green; ex. 488 nm; em. 510-545 nm) and NiNTA-PE DiD lipid bilayer (blue: ex. 633 nm; em. 650-750 nm). In order to determine the surface density of N-Cadherin on the proteolipobeads, Quantum FITC MESF premix kit was performed. Quantum FITC MESP premix kits are used in the quantitation of FITC fluorescence intensity in Molecules of Equivalent Soluble Fluorochrome (MESF) units (Bangs Laboratories). The kit allows the direct quantitation of the fluorescence intensity of a sample in terms of MESF units, which were converted from the flow cytometry results. FITC MESF kits were comprised of 5 populations of calibrated FITC fluorescent standards, 4 populations of different levels of FITC fluorescent microspheres and 1 blank population. The FITC MESF kits have excitation and emission spectra matching those proteolipobeads labeled with FITC. Using Anti N-Cadherin FITC allows us estimate the N-Cadherin binding levels in MESF units. ImageJ 1.43 was used in all image analysis using the following plugins: Mander's coeeficients, 3D particle counter.

### N-Cadherin Surface Density Characterization

Proteolipobeads were incubated with FITC conjugated antibody at 1:500 at 4° C. overnight after 1 hour 0.1% BSA blotting, at greater than 2-fold excess. The Ni-NTA-PE DiD beads without N-Cadherin binding were used as positive control. And the 30 micron beads were chosen as negative control to test non-specific binding. Quantum FITC MESF kit was used in the quantitation of FITC fluorescence intensity in Molecules of Equivalent Soluble Fluorochrome (MESF) units. This kit allows the quantitation of antibody binding capacity. The standard FITC beads helped us establish a calibration curve of 5 fluorescence intersity populations. Analyze the proteolipobeads along with the positive and negative control separately and record each samples FITC fluorescence intensity peak. Finally use the calibration plot to determine the MESF value that corresponds to each peak.

### Hybrid Matrix Construction and Characterization

Rat-tail collagen type I was purchased from Becton Dickenson Lab with an original concentration of 5 mg/ml. Collagen was immediately neutralized by 10× PBS and 1N NaOH on ice and diluted into final concentration of 0.5 mg/ml. Before the hMSCs were loaded into this system, proteolipobeads were suspended into the collagen gel and mixed well. After mixing, transfer the solution immediately to 37° C. incubator for 30-60 minutes to initiate self-assembly of the collagen. 3D collagen matrix was imaged by scanning electron microscopy and confocal microscopy 3D reconstruction scanning.

### MSC Loading

Stem cells were suspended in medium with an original concentration of 5×10⁶ cells/ml, which were mixed with the neutralized collagen solution with final cell densities of 1×10⁵. The cell mixture was dispensed as 2-5 microliter droplets by thin needle glass syringe onto a collection flatform of sterilized non-adherent parafilm surface. The droplets formed solid gel microspheres after incubating at 37° C. water bath for 30-60 minutes, which were then gently flushed with full medium into a mini Petri dish with 0.15 mm cover slip located in the middle for later confocal microscope imaging.

### CLSM and FRAP and Data Analysis of Cell-PLB Interactions

CLSM sequential scanning was performed after two hours MSC loading into collagen matrix as day zero data and 24 hours as day one, 96 hours as day four. In sequential scan mode, green, red, and blue images were recorded line by line in a sequential order instead of acquiring them in simultaneously, effectively eliminating crosstalk or spectral bleedthrough between channels. The confocal settings were designed to optimize performance and image quality of the 3D data sets. Reconstruction of confocal 3D scanning images showed cell-proteolipobead interactions and the coverage of N-Cadherin was obtained by the surface area covered by the MSCs to the total proteolipobead surface area ratio.

While the invention has been described with reference to certain embodiments, it will be understood by those skilled in the art that various changes may be made and equivalents may be substituted for elements thereof to adapt to particular situations without departing from the scope of the disclosure. Therefore, it is intended that the claims not be limited to the particular embodiments disclosed, but that the claims will include all embodiments falling within the scope and spirit of the appended claims.

What is claimed is:

1. A method of delivering a compound to a target biological cell, the method comprising the steps of:
exposing a target biological cell to a proteolipobead, the proteolipobead comprising:
a discrete particle providing a core to the proteolipobead;
a phospholipid bilayer surrounding the discrete particle in three-dimensions;
a compound suspended in the phospholipid bilayer such that the compound can undergo lateral motion within the phospholipid bilayer;
permitting the compound to be transferred from the phospholipid bilayer to the target biological cell.

2. The method as recited in claim 1, wherein the compound is a protein.

3. The method as recited in claim 1, wherein the discrete particle has a diameter of at least about 10 micrometers and less than about 100 micrometers.

4. The method as recited in claim 1, wherein the discrete particle has a diameter sufficient to prevent the proteolipobead from being absorbed by the target biological cell.

5. The method as recited in claim 1, wherein the discrete particle is comprised of a material selected from the group consisting of silica, glass, and a magnetic material.

6. The method as recited in claim 1, wherein the discrete particle is substantially spherical.

7. The method as recited in claim 1, wherein the phospholipid bilayer comprises a cell adhesion peptide selected from the group consisting of an integrin, a selectin, and a cadherin.

8. The method as recited in claim 1, wherein the phospholipid bilayer comprises a cell adhesion peptide cadherin.

9. The method as recited in claim 1, wherein the proteolipobeads are disposed in a hydrogel, the method further comprising co-culturing the target biological cell in the hydrogel with the proteolipobeads.

10. The method as recited in claim 9, wherein the target biological cell is a stem cell.

11. The method as recited in claim 10, wherein the target biological cell is a human mesenchymal stem cell.

12. The method as recited in claim 1, wherein the phospholipid bilayer comprises a diglyceride phospholipid and a non-polar organic molecule.

13. The method as recited in claim 12, wherein the non-polar organic molecule is cholesterol.

14. The method as recited in claim 12, wherein the diglyceride phospholipid is 1-palmitoyl-2-oleoyl-sn-glycero-3-phosphatidylcholine (POPC).

15. The method as recited in claim 12, wherein the phospholipid bilayer further comprises a salt of 1,2-di-(9Z-octadecenoyl)-sn-glycero-3-[(N-(5-amino-1-carboxypentyl)iminodiacetic acid)succinyl].

16. The method as recited in claim 1, wherein the discrete particle is functionalized and the phospholipid bilayer is tethered to the discrete particle.

17. The method as recited in claim 1, wherein the discrete particle is coated with a polymeric material and the phospholipid bilayer surrounds, but is no covalently bound to, the polymeric material.

18. A composition of matter that forms a proteolipobead upon exposure to an aqueous solution, the composition of matter comprising:
a discrete particle providing a core to the proteolipobead;
a phospholipid that forms a bilayer surrounding the discrete particle in three-dimensions when the composition is exposed to the aqueous solution;
a peptide that is suspended in the phospholipid bilayer when the composition is exposed to the aqueous solution such that the peptide can undergo lateral motion within the phospholipid bilayer.

19. The composition of matter of claim 17, wherein the composition is dehydrated.
